(19) United States
(12) Patent Application Publication
(10) Pub. No.: US 2016/0016990 A1
(43) Pub. Date: Jan. 21, 2016

(54) PROTEIN PURIFICATION BY MEANS OF AQUEOUS TWO-PHASE CENTRIFUGAL EXTRACTION

(71) Applicant: BOEHRINGER INGELHEIM INTERNATIONAL GMBH, Ingelheim Am Rhein (DE)

(72) Inventors: Michael RICHTER, Ummendorf (DE); Michael DIETERLE, Biberach an der Riss (DE); Frederik RUDOLPH, Biberach an der Riss (DE)

(21) Appl. No.: 14/773,143

(22) PCT Filed: Feb. 26, 2014
(86) PCT No.: PCT/EP2014/053768

§ 371 Date: Sep. 4, 2015

(30) Foreign Application Priority Data
Mar. 8, 2013 (EP) 13158285.0

(51) Int. Cl. C07K 1/14; B01D 21/26; C07K 16/00

(52) U.S. Cl. CPC C07K 1/145; C07K 16/00; B01D 21/262

(57) ABSTRACT

The invention relates to a method for the selective purification and concentration of immunoglobulins or other proteins by means of an aqueous two-phase system using a centrifugal extractor.

### BACKGROUND TO THE INVENTION

1. Technical Field

The present invention relates to methods for purifying proteins, particularly immunoglobulins.

2. Prior Art

Biomolecules such as proteins, polynucleotides, polysaccharides and the like have increasingly been gaining commercial significance as medicaments, as diagnostic agents, as additives for foodstuffs, as detergents and the like, as research reagents and for many other applications. The need for such biomolecules cannot generally be satisfied by isolating the molecules from natural sources—e.g. in the case of proteins—but requires the use of biotechnological production methods.

The biotechnological production of proteins typically begins with cloning a DNA fragment into a suitable expression vector. After transfection of the expression vector into suitable prokaryotic or eukaryotic expression cells and subsequent selection of transfected cells the latter are cultivated in fermenters and the desired protein is expressed. Then the cells or the culture supernatant are harvested and the protein contained therein is worked up and purified.

In the case of eukaryotic expression systems, i.e. when using mammalian cell cultures such as CHO— or NS0- cells, in the last 15 years there has been a significant increase in the concentration of the desired protein which can be achieved in the cell cultures or cell culture supernatants in the expression step (1). Over the same period the binding capacity of chromatography materials which are used during the subsequent purification of the proteins has improved only slightly. For this reason there is an urgent need for improved, optimised purification processes for biomolecules, particularly proteins, which can be carried out on a large industrial scale (2).

In the case of biopharmaceuticals, such as proteins used as medicaments, e.g. therapeutic antibodies, in addition to the product yield the removal of impurities is also of outstanding importance. A distinction can be drawn between process-dependent impurities and product-dependent impurities (3). The process-dependent impurities contain host cells as well as components of the host cells such as proteins (“host cell proteins”, HCP) and nucleic acids. These come from, inter alia, the cell culture (such as media ingredients) or from the working up (such as salts or detached chromatography ligands). Product-dependent impurities are molecular variants of the product with differing properties (4). These include shortened forms such as precursors and hydrolytic breakdown products, but also modified forms produced for example by deamination, incorrect glycosylations or incorrectly inked disulphide bridges. The product-dependent variants also include polymers and aggregates. Further impurities are contaminants. The term is used to denote all other materials of a chemical, biochemical or microbiological nature which do not directly belong to the manufacturing process. Examples of contaminants include viruses which may undesirably occur in cell cultures.

Impurities lead to safety concerns in the case of biopharmaceuticals. These are intensified if, as is very often the case in biopharmaceuticals, the therapeutic proteins are administered by injection or infusion directly into the bloodstream. Thus, host cells and host cell components may lead to allergic reactions or immunopathological effects. In addition, impurities may also lead to undesirable immunogenicity of the protein administered, i.e. they may trigger an undesirable immune response by the patient to the therapeutic agent, possibly to the point of life-threatening anaphylactic shock. Therefore, there is a need for suitable purification processes by means of which all undesirable substances can be depleted to an insignificant level.

On the other hand, economic aspects cannot be ignored in the case of biopharmaceuticals. Thus, the production and purification methods used must not jeopardise the economic viability of the biopharmaceutical product thus produced.

In biopharmaceuticals, a number of process steps are used for working up proteins from cell cultures. First, the host cells are separated from the cell culture using a separator. Then, initial impurities and turbidity as well as final host cells are removed by means of a filter cascade (deep filter and clarifying filter as well as a sterile filter). Charged molecules such as DNA, HCP and turbidity are retained in the filter materials (2). This cell culture supernatant can then be isolated with maximum purity in further working-up steps using chromatography columns and filters.

However, harvesting host cells is time-consuming and expensive and there are limitations to filtration, particularly with increasing product concentrations in the fermenter and large amounts of product. The critical points are: loading capacities, process pressure caused by turbidity and hence long processing times (5).

In addition to filtration and precipitation steps, (column-chromatographic) methods are of central importance as steps for working up and purifying proteins. Thus, the concentration of antibodies frequently includes a step of purification by affinity chromatography. Accordingly, nowadays, there are numerous known column chromatographic methods and chromatography materials which can be used with them.

Affinity chromatography matrices are used as the stationary phase in the industrial purification of various substances (6). By means of immobilised ligands, it is possible to specifically concentrate and purify substances that have a certain affinity for the particular ligand used. For the industrial purification of antibodies (immunoglobulins), particularly the purification of monoclonal antibodies, immobilised protein A is often used as the initial purification step. Protein A is a protein with about 41 kDA of Staphylococcus aureus, that binds with high affinity (10⁻⁸ M-10⁻¹² M of human IgG) to the CH₂/CH₃— domain of the Fc- region of immunoglobulins. In protein A chromatography immunoglobulins or fusion proteins that have a protein-A-binding Fc- region from the mobile phase bind specifically to the protein A ligand, which is covalently bound to a carrier (e.g. Sepharose). Protein A from Staphylococcus aureus (wild-type protein A) and genetically modified recombinant protein A (rec. protein A) interacts, via non-covalent interactions, with the constant region (Fc- fragment) of the antibodies (7). This specific interaction can be utilised to separate impurities efficiently from the antibody. By modifying the pH the interaction between antibody and protein A ligand can be deliberately stopped and the antibody can be released or eluted from the stationary phase.

However, affinity chromatography is expensive and, precisely when there are increasing concentrations of product in the fermenter and large amounts of product, chromatographic purification processes come up against limitations. The critical points are: loading capacities, number of cycles, processing times, pool volumes and amounts of buffer. Alternative purification methods are therefore essential for the future. A general overview of conventional purification strategies as well as affinity chromatography and alternative methods of chromatography is provided in the following articles (8; 9).

For some years, pharmaceutical research and development has been working more intensively on new biopharmaceuticals which do not have an Fc- region (10). For these non-antibodies or proteins there have up till now been few suitable chromatography matrices on the market. Prices for optimised matrices (loading capacity) and the development time for processes with new matrices will climb (11).

Fermentation is a volume-related process, i.e. an increase in the product concentration does not have a direct influence on the process as the same apparatus, such as fermentation reactors, can be used. In most conventional protein purification processes, adsorption- and filtration-based techniques are used. These are mass-related in their operation and therefore have to purify larger quantities of product at increasing product concentrations and may then come up against their loading limits. As these techniques cannot be upscaled at will, or are very expensive to use, the use of alternative techniques is increasingly being considered (12).

One highly promising alternative to the adsorption- and filtration-based techniques mentioned hereinbefore is the gentle extraction of biomolecules (proteins, nucleic acids, cells, etc.) by means of aqueous two-phase systems (ATPS). ATPS consists of an aqueous solution with two immiscible polymers or one polymer and an inorganic salt. After the above-mentioned components have been mixed, two phases are formed in the system. On the basis of different biophysical properties, the biomolecules are distributed between the two phases. The concentration and purification of the target molecule can thus be carried out in one process step. Polyethyleneglycol (PEG) is one of the polymers most frequently used for phase formation (19).

The aqueous two-phase system was proposed for the first time in 1957. Since then, aqueous two-phase systems have been used for the purification of a variety of proteins and also plasmid DNA (13; 14; 15). To achieve higher purity of the target molecule, multi-stage ATPS extractions may be carried out. Thus, for example, the purity of immunoglobulin G of 43% after one-step ATPS extraction was able to be improved to 93% by a 4-step ATPS extraction (16).

However, right up to the present day, there is not a sufficient understanding of the process to enable ATPS to be used industrially. For this, process parameters have to be evaluated and scale-up data have to be obtained, while phase-building components also have to be more closely characterised.

In order to separate aqueous two-phase systems, extraction apparatus such as, for example, plate columns or mixer settlers have to be used (17).

The plate column used for the extraction of liquid phases consists of two separators and a central cylindrical part. The separators, which are located at the bottom and top of the column, contain an inlet and outlet for the light and heavy phases, respectively. A pulsing system located on the base of the central median part allows uniform distribution and mixing of the two phases throughout the cross-section of the column and thus provides conditions for an exchange of substances between the two phases.

The mixer-settler apparatus consists of a mixing chamber and a settling chamber. In the mixing chamber, the two phases are aspirated through a speed-controlled stirrer and dispersed. After thorough mixing the liquid is transferred into the settling chamber for settling out the two phases.

Another technical apparatus for separating two immiscible liquids is the centrifugal extractor.

In a centrifugal extractor, two immiscible liquids with different densities are fed into a pump chamber. The liquid/liquid mixture is drawn into the centrifuge flask through a pump turbine located on the base of the rotating flask. The liquids are separated by centrifugal force which is produced by the rotating flask. The heavy phase accumulates in the outer part of the centrifuge flask. The light phase accumulates in the inner part. The position of the liquid/liquid interphase is regulated by a so-called heavy-phase weir. Exchangeable heavy-phase weirs with different diameters offer the possibility of processing different density ratios of the liquids. The heavy phase is taken up through a stationary chamber and released through an outlet. The light phase is also taken up in a separate stationary chamber and released through another outlet for further processing.

In a liquid/liquid centrifugal extractor, three process parameters can be set: the loading or pumping flow, the speed of rotation of the centrifuge flask and the interphase position.

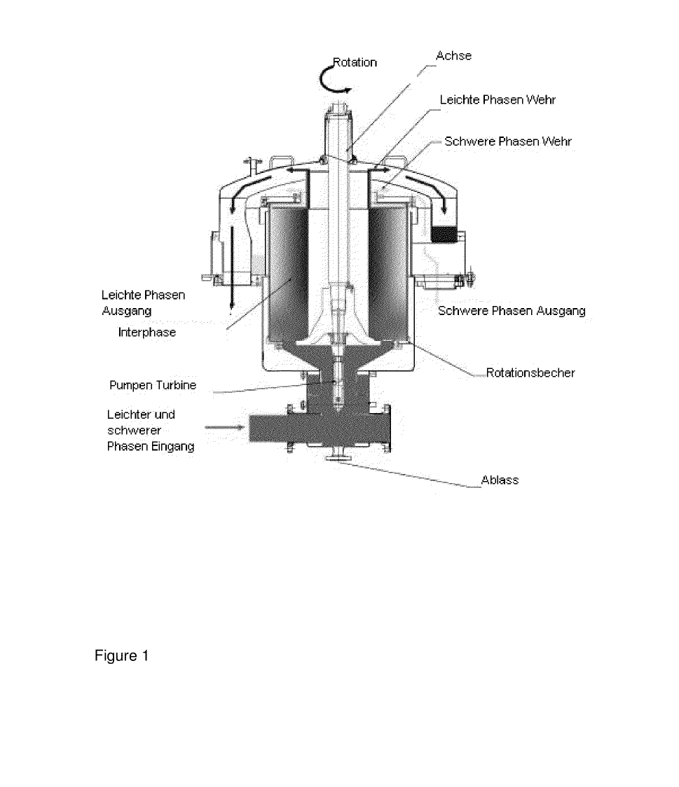

FIG. 1 is a schematic representation of a centrifugal extractor manufactured by Rousselet-Robatel.

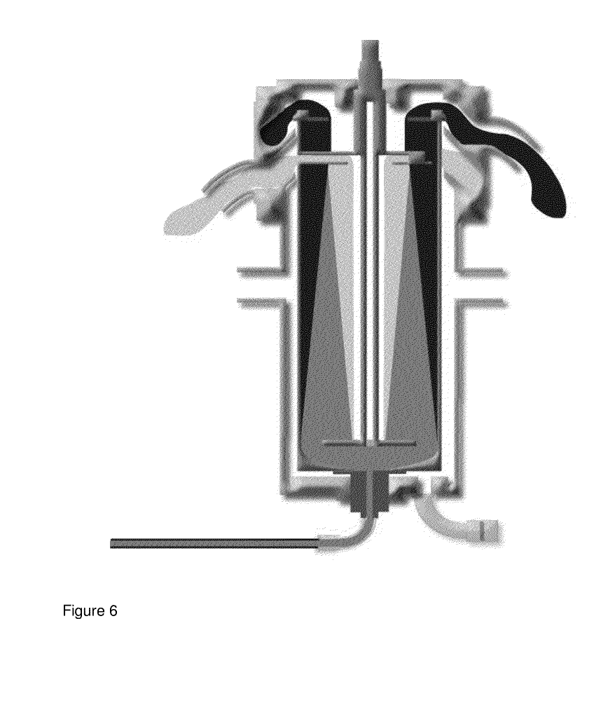

FIG. 6 shows the principle of a centrifugal extractor operating on the “Direct Feed” principle (source: Wikipedia/Vornefeld; http://commons.wikimedia.org/wiki/File:Sep-DF-7.jpg). In “Direct feed” the mixed liquids are fed through the base plate directly into the rotor and thus the shear forces on the product are minimised. The two phases are continuously separated into a heavy phase and a light phase.

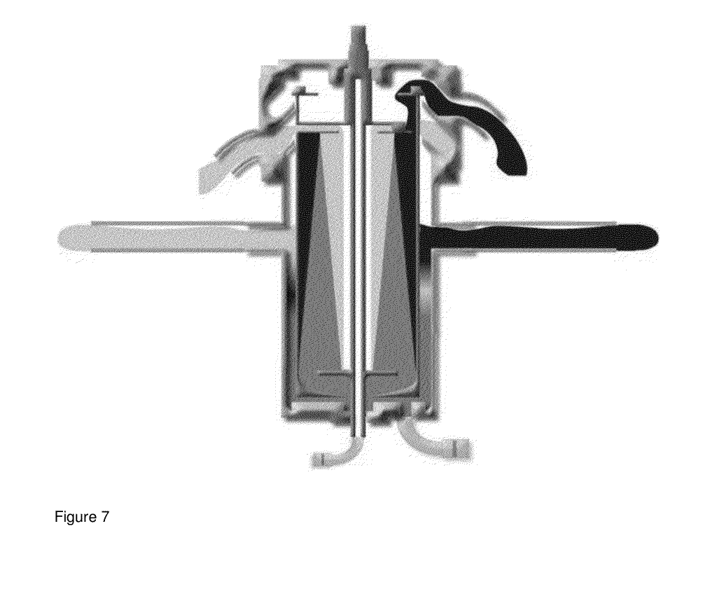

FIG. 7 shows the principle of a centrifugal extractor according to the “mix and sep” principle (source: Wikipedia/Vornefeld, http://commons.wikimedia.org/wiki/File:Mixsep.jpg). In “mix & sep” two immiscible liquids of different densities are brought into contact outside the rotor. As a result of the shear forces produced in the narrow gap between the rotating rotor and the static housing wall, very small drops with large surface areas are formed for the ideal transition of the substance from one phase into the other. Then the two phases are separated from one another in the rotor.

### BRIEF SUMMARY OF THE INVENTION

The present invention is a combination of cell harvest, protein separation and DNA depletion from cell cultures by means of aqueous two-phase systems and using a centrifugal extractor. The invention completely dispenses with the conventional use of separator, filtration and chromatography.

The invention described here makes use of aqueous two-phase separation (extraction) instead of the process steps for the separation and filtration of host cells and affinity chromatography for purifying biomolecules. The extraction is carried out using a centrifugal extractor. This is able to perform the extraction or separation of the phase systems used into light and heavy phases on a production scale of for example 12000 L of cell culture in a few hours. Processing of the same volume in the mixer settler described above or a plate column would take several days. Using a centrifugal extractor, aqueous two-phase extractions can be processed 50-250 times faster, compared with separation by alternative methods such as the plate column or the mixer settler.

Using the method according to the invention, proteins can be concentrated stably and with no aggregation.

In one aspect, the invention relates to a method for the selective purification and accumulation of immunoglobulins or other proteins using an aqueous two-phase system comprising the steps of
a. preparing a cell culture or a cell culture supernatant which contains the target protein;
b. converting the cell culture or the cell culture supernatant into an aqueous two-phase system by the addition of a polymer and at least one salt, or two polymers in a suitable concentration;
c. thoroughly mixing the two-phase system to produce a dispersion;
d. separating the heavy and light phases in a centrifugal extractor;
e. recovering the target protein from the light phase.

By “purification and accumulation” is meant that unwanted components such as cells, DNA and host proteins can be depleted and the purity of the target protein is increased by the process.

In another aspect the invention relates to a method for the selective purification and accumulation and additional concentration of immunoglobulins or other proteins by means of an aqueous two-phase system, comprising the steps of
a. preparing a cell culture or a cell culture supernatant which contains the target protein;
b. preparing a light phase of the aqueous two-phase system by adding a polymer and at least one salt, or two polymers, to an aqueous medium in a suitable concentration;
c. converting the cell culture or the cell culture supernatant into the heavy phase of the aqueous two-phase system by the addition of a polymer and at least one salt, or two polymers in a suitable concentration;
d. separately feeding the light and heavy phases into the mixing chamber of the centrifugal extractor at different flow rates, the ratio of the flow rates corresponding to the desired accumulation ratio;
e. thoroughly mixing the two-phase system to produce a dispersion;
f. separating the heavy and light phase in the centrifugal extractor;
g. recovering the target protein from the light phase.

By “concentration” is meant, in this context, that as the result the target protein is present in a higher concentration (mass/volume). In the light phase from which it can be recovered by the process, it is present in a higher concentration than in the cell culture or the cell culture supernatant or in the heavy phase in which it is fed into the centrifugal extractor.

By “recovering the target protein from the light phase” is meant that the light phase with the target protein contained therein is processed further in order to obtain the target protein in the form in which it is finally desired. This may involve further purification steps, concentration steps and/or changing the medium e.g. by re-buffering or removing/adding other substances.

In one aspect of the method, the two-phase system comprises polyethyleneglycol with a molecular weight of between 200 and 1000 g/mol.

In one aspect of the method, the two-phase system contains polyethyleneglycol with a molecular weight of between 400 and 600 g/mol.

In one aspect of the method, the polyethyleneglycol is present in the two-phase system in a concentration of 18 to 35% by weight.

In one aspect of the method, the salt or one of the salts is a phosphate salt.

In one aspect of the method, the pH of the two-phase system is between 5 and 7.

In one aspect of the method, the centrifugal extractor is pre-equilibrated with heavy phase.

In one aspect of the method the two phases are separated at a centrifugal acceleration within a range from 90-500×g.

In one aspect of the method, the two phases are separated at a centrifugal acceleration within a range from 90-450×g.

In one aspect of the method, the two phases are separated at a centrifugal acceleration within a range from 40-100% of the maximum possible acceleration in the apparatus used.

In one aspect of the method, the density ratio of the two phases is at least 1.06.

### DETAILED DESCRIPTION OF THE INVENTION

As stated hereinbefore, ATPS consists of an aqueous solution with two immiscible polymers, or one polymer and an inorganic salt. After the above-mentioned components have been mixed, two phases are formed in the system. As a result of their different biophysical properties, the biomolecules go into one of the two phases.

For separating host cells, DNA and proteins from cell culture using an aqueous two-phase system, suitable extraction systems are required in which the host cells and the DNA accumulate more in one phase and the target protein accumulates in the other, second, phase. Depending on the protein stability, the protein may accumulate in the light and heavy phase. Separation is frequently carried out using PEG phosphate systems in different concentrations. The PEG (polyethyleneglycol) accumulates in the upper light phase. The phosphate accumulates in the heavy lower phase. Proteins have only limited stability in PEG and phosphate and are precipitated in high concentrations of PEG and phosphate. As high concentrations are required to form two-phase systems, not every PEG and phosphate system can be used for the separation. PEG is available in different molecular weights. PEG with molecular weights>1000 g/mol have a tendency to precipitate the protein even at low concentrations. For extractions in which the protein should accumulate in the light PEG phase, PEG with a molecular weight<1000 g/mol should therefore be used. When PEG with a molecular weight of below 1000 g/mol is used, it is also unnecessary to add an additional salt (e.g. common salt) to the two-phase system in order to achieve the accumulation in the light phase as described in the prior art (16).

As high concentrations of phosphate are needed to form aqueous two-phase systems by means of PEG400 (400 g/mol) and PEG600 (600 g/mol), systems of this kind are particularly suitable for processing/separation in a centrifugal extractor.

The advantage of a centrifugal extractor as opposed to a plate column and a mixer settler is that it can separate the light and heavy phases of stable aqueous extraction systems in a few seconds. This is made possible by centrifugal force, in contrast to the sedimentation in mixer settler apparatus, for example. To achieve fast extraction times, suitable systems must be selected. The short separation time of the light and heavy phases is primarily dependent on the great difference in density between the light and heavy phases. Transition times of 2-10 s for the proteins from the heavy phase into the light phase are made possible by density ratios of light to heavy phase 1.18.

In PEG400 and PEG600 phosphate systems with a pH range from 5 to 7, the proteins are particularly stable. Systems with the following proportions of PEG and phosphate in the light phase (PEG phase) are suitable for the high-yield separation of proteins using aqueous two-phase systems.

PEG400/phosphate pH 5 to 7:

With 18-35 wt. % PEG400 and 8-15 wt. % phosphate

PEG600/phosphate pH 5 to 7:

With 18-35 wt. % PEG600 and 6-15 wt. % phosphate

Thanks to their density ratios 1.06, the PEG/phosphate systems used can enable rapid process times while at the same time achieving a high protein stability in the uptake phase of the PEG phase.

The particular PEG/phosphate system is weighed out or prepared. Meanwhile the cell culture is added to the aqueous phase system. The aqueous phase system is mixed using a stirring apparatus. The phosphate salts used may be for example alkali metal hydrogen phosphates such as sodium dihydrogen phosphate or potassium monohydrogen phosphate. The centrifugal extractor is advantageously first equilibrated with the heavy phase. The rotor may be set to a centrifugal acceleration in the range from 40-100% of the maximum possible g-force (depending on the equipment). Preferably, a centrifugal acceleration of 90-450×g may be applied, corresponding to a revolution speed of between 2000 and 4500 rpm in the centrifugal extractor model BXPO40 made by Rousselet Robatel, taken as an example. At the same time, the heavy phase is introduced into the centrifugal extractor by means of a pump. When only the heavy phase added is still flowing out of the heavy phase outlet, the aqueous phase mixture can be added with the cell culture at the same pumping rate and rotation speed.

The light phase (PEG phase) with the target protein is then recovered through the outlet for the light phase, while the host cells, DNA and fragments thereof accumulate in the heavy phase.

In another aspect of the invention, in addition to the purification and accumulation of the target protein, it is also possible to concentrate the target protein, i.e. the concentration of the target protein in the light phase can be increased compared with the concentration in the cell culture. To do this, another process control operating on the “mix & sep” principle can be used. In this, the cell culture is added to a heavy phase with a high phosphate concentration and a low PEG concentration (e.g. 18-39 wt. % phosphate and 10.5-0.2 wt. % PEG). A light phase (protein uptake phase) with a low phosphate and a high PEG concentration is prepared separately (e.g. 2-10 wt. % phosphate and 43.8-21.2 wt. % PEG). The centrifugal extractor is advantageously first equilibrated with the heavy phase, as described above. When only the added heavy phase is still flowing out of the heavy phase outlet, the light phase can then be introduced into the centrifugal extractor by means of a separate pump. The heavy phase (with cell culture) is introduced into the separator at high flow rates (e.g. 6-25 kg/h for the centrifugal extractor BXPO40 used) and the light phase (uptake phase) is introduced into the separator at low flow rates (e.g. 0.3-3 kg/h for the centrifugal extractor BXPO40 used).

The ratio of the pumping flows of heavy phase through the light phase corresponds to the concentration ratio of the protein solution and is preferably 5 to 50 times (5-50×). In the centrifugal extractor the light and heavy phase are mixed in the mixing chamber (centrifuge flask).

The light phase (PEG phase) with the concentrated target protein is then obtained through the light phase outlet, while the host cells, DNA and fragments thereof accumulate in the heavy phase. Because of the lower pumping flow of the light phase, the volume of the protein uptake phase obtained from the centrifuge is less than that of the heavy phase and the target protein is concentrated accordingly.

### EXAMPLES

Materials and Methods:

Phosphate Stock Solution:

To prepare 1 L of a 40 wt. % phosphate stock solution, 262.1 g of Na-dihydrogen phosphate (NaH₂PO₄×2 H₂O) and 198.4 g potassium monohydrogen phosphate K₂HPO₄) are added to 539.5 g H₂O and mixed homogeneously.

PEG400:

Polyethyleneglycol with an average molecular weight of 400 g/mol

Phase System A (for Preparing the Equilibrating Solution)

In order to prepare phase system A, PEG400 and phosphate stock solution as well as sodium chloride (NaCl) is adjusted to the following concentrations with H₂O. 23 wt. % PEG400, 14 wt. % phosphate and 5 wt. % NaCl, pH 6

Phase System B with Cell Culture

In order to prepare phase system B, PEG400 and phosphate stock solution as well as sodium chloride (NaCl) is adjusted to the following concentrations with a cell culture. 23 wt. % PEG400, 14 wt. % phosphate stock solution and 5 wt. % NaCl, pH 6

Phase System C (for Preparing the Equilibrating Solution)

In order to prepare phase system A, PEG400 and phosphate stock solution is adjusted to the following concentrations with H₂O.

23 wt. % PEG400, 14 wt. % phosphate, pH 6

Phase System D with Cell Culture

In order to prepare phase system D, PEG400 and phosphate stock solution is adjusted to the following concentrations with cell culture and H₂O.

23 wt. % PEG400, 14 wt. % phosphate stock solution, pH 6

Phase System E with Pre-Purified Protein

In order to prepare phase system E (heavy phase), PEG400 and phosphate stock solution is adjusted to the following concentrations with protein and H₂O.

1.25 wt. % PEG400, 31 wt. % phosphate stock solution, pH 6, and 3.3 wt. % NaCl.

Eukaryotic Cell Culture
BIMAB19a. The cell culture of the CHO— cells with impurities such as DNA and host cells as well as target protein is obtained after several days' culturing.

(Experiment 1)
BIFAB39. The cell culture of the CHO— cells with impurities such as DNA and host cells as well as target protein is obtained after several days' culturing.

(Experiment 3)

Cell Culture Supernatant

The cell culture supernatant of CHO— cells with impurities such as DNA and target protein is obtained after several days' culturing by filtration or centrifugation.

The concentration of target protein BIMAB01 is determined by means of Protein A analysis as 1.29 mg/ml.

Pre-Purified Protein

The purified protein BIMAB01 is obtained after a multi-stage purification process (affinity chromatography+anion exchange, cation exchange, filtration).

UV Detection

In order to determine the antibody content of purified protein, UV detection is also carried out at 280 nm according to the Beer-Lambert law.

Protein A HPLC

In order to determine the antibody content of cell culture-free supernatant and of purified antibody, a protein A HPLC on a Waters or Dionex apparatus was used (injector pumps and column oven W2790/5, UV detector W2489). The antibody content of the solutions was determined by means of the UV signal of the acidic eluting peak.

Biacore Measurement of Content

The protein content of FAB39 is determined using the biospecific interaction analysis on the Biacore system (BIA=biospecific/biomolecular interaction analysis). The concentration is calculated relative to a standard curve by means of the 4-parameter function of the Biacore Control Software.

Biacore Measurement of Activity

In order to determine the activity, the binding activity of FA39 to its target molecule in relation to the corresponding reference material is determined. This is done using the biospecific interaction analysis on the Biacore system (BIA=biospecific/biomolecular interaction analysis). The activity is calculated as the quotient of the active concentration (sample, compared to the reference material) and the total protein content (determined by means of UV scan, protein A/G—Biacore, or comparable) in percent [%].

Density Measurement

The density of the light and heavy phase was measured using the densitometer DM40 made by the company Mettler Toledo. Measurements were carried out according to the manufacturer's instructions.

Determination of Cell Count

The cell count was determined with a Fuchs Rosenthal counting chamber and the Olympus BH2 microscope. The cells had previously been stained with a trypan blue solution.

Centrifugal Cxtractor

The model BXPO40 made by Rousselet Robatel with a useful volume of 0.11 L and a nominal throughput of 50 L/h was used.

Pump (Hose Pump)

Watson Marlow 503S for supplying the phase system and the equilibrating solution.

Experiments:

1^{st} experiment: DNA and cell depletion from cell culture using centrifugal extractor

In order to separate host cells, DNA and target protein from a cell culture using aqueous two-phase extraction, first of all a phosphate stock solution is prepared. Then the phase system A is adjusted by weighing to the concentrations stated above. The phase system A is introduced into a separating funnel, to obtain the equilibrating solution of the centrifugal extractor (pure heavy phase).

After two phases with a stable volume have formed in the separating funnel, the light (upper) and heavy (lower) phases (equilibrating solution) are separated and recovered separately.

At the same time the phase system B with cell culture is prepared. This phase system is homogeneously mixed using a stirrer.

The interphase (diameter of heavy-phase weir—diameter of light phase weir) was regulated to 2 mm by the incorporation of a heavy-phase weir.

Then, the equilibrating solution is introduced into the centrifugal extractor through the heavy phase inlet by means of a pump with a high pumping rate; this serves to equilibrate the centrifugal extractor. To do this, the centrifuge flask is accelerated to a rotation speed of between 2500-3500 RPM. When only the added equilibrating solution is still flowing out of the heavy phase outlet of the centrifugal extractor, phase system B (with cell culture) can then be fed in through the heavy phase inlet at the same pumping rate and rotation speed.

Light phase (PEG phase) with the target protein is then obtained through the outlet for the light phase, while the host cells, DNA and fragments thereof accumulate in the heavy phase.

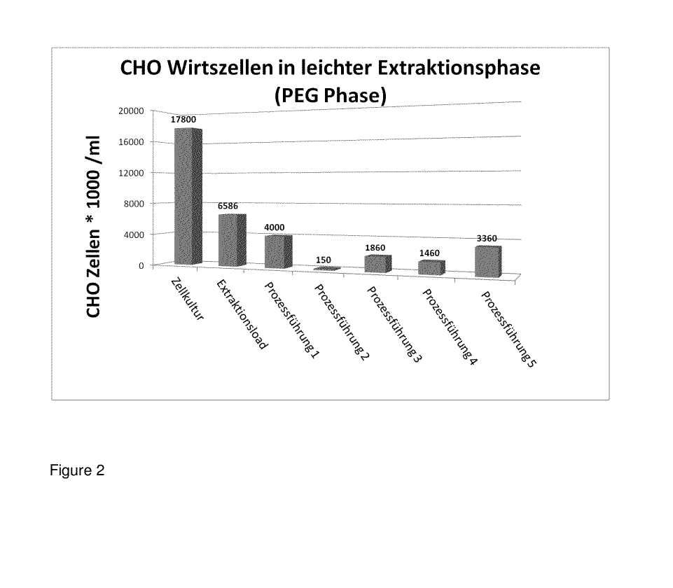

FIG. 2 shows a measurement of the number of CHO host cells in the light phase after extraction in the centrifugal extractor under different process controls.

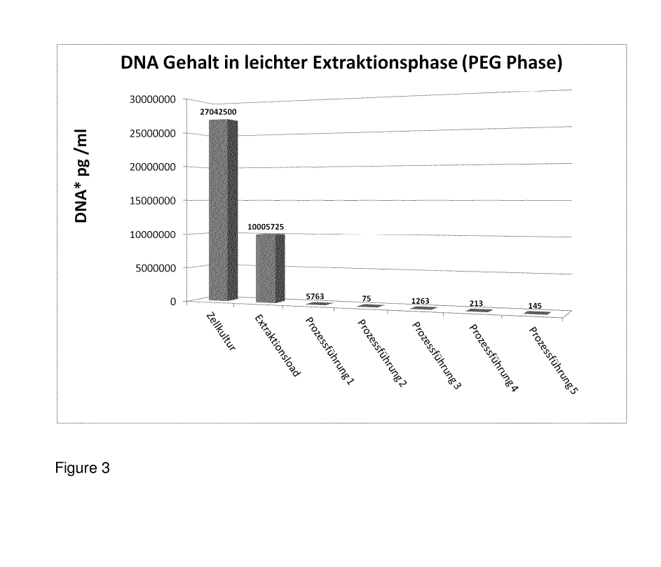

FIG. 3 shows the DNA content in the light phase after extraction in the centrifugal extractor under different process controls.

TABLE 1
Process strategies in the centrifugal extractor

| | Pump flow | Rotation of centrifuge flask |
|---|---|---|
| Strategy | L/H | RPM |
| process control 1 | 8.6 | 2500.0 |
| process control 2 | 22.8 | 2500.0 |
| process control 3 | 5.7 | 3500.0 |
| process control 4 | 22.8 | 3500.0 |
| process control 5 | 22.8 | 3000.0 |

2^{nd} experiment: DNA depletion from cell culture supernatant by means of a centrifugal extractor

In order to separate DNA and target protein from a cell culture supernatant using aqueous two-phase extraction, first of all a phosphate stock solution is prepared. Then the phase system A is prepared by weighing to the correct concentrations as described above. The phase system A is placed in a separating funnel. After two phases with a stable volume have formed in the separating funnel, the light (upper) and heavy (lower) phases (equilibrating solution) are separated and recovered separately.

Using the same method, the phase system B with cell culture supernatant is prepared. It is mixed homogeneously with a stirrer. 1750 g phosphate stock solution, 1150 g PEG400, 250 g NaCl and 1850 g cell culture supernatant are weighed in. This results in a protein concentration in phase system B of 0.48 g/L and a DNA concentration of 2941473 pg DNA /mg of protein.

The interphase in the centrifugal extractor (diameter of heavy-phase weir—diameter of light phase weir) is regulated to 2 mm by incorporation of a heavy-phase weir. Then, 200 g of the equilibrating solution is pumped into the centrifugal extractor through the heavy phase inlet by means of a pump at 22.8 L/H. To do this, the centrifuge flask is accelerated to a rotation speed of 3000 RPM.

When only the added equilibrating solution is still flowing out of the heavy phase outlet of the centrifugal extractor, 4077 ml of phase system B (with cell culture supernatant) are fed in through the heavy phase inlet at the same pumping rate and rotation speed. Light phase (PEG phase) with the target protein is then obtained from the light phase outlet. DNA and fragments thereof accumulate in the heavy phase.

TABLE 2
Results of Experiment 2

| | |
|---|---|
| Total phase system B [ml] | 4077 |
| Conc. protein in ATPS [mg/ml] | 0.48 |
| Total protein [mg] | 1945 |
| Total DNA content ATPS [pg DNA/mg protein] | 2941473 |
| Volume of light phase [ml] | 2573 |
| Conc. MAB light phase [mg/ml] | 0.75 |
| Protein in light phase [mg] | 1930 |
| Yield of MAB light phase [%] | 99.2 |
| DNA content of light phase [pg DNA/mg protein] | 144 |
| DNA depletion [log stage] | 4.3 |
| Volume of heavy phase [ml] | 1148 |
| Conc. MAB heavy phase [mg/ml] | 0.01 |
| Protein in heavy phase [mg] | 11 |
| DNA content [pg DNA/mg Protein] | 401200000 |

3^{rd} experiment: DNA and cell depletion from cell culture by means of centrifugal extractor

In order to separate host cells, DNA and target protein from a cell culture by aqueous two-phase extraction, first of all a phosphate stock solution is prepared. Then the phase system C is adjusted to the above-mentioned concentrations by weighing. The phase system C is placed in a separating funnel in order to obtain the equilibrating solution of the centrifugal extractor (pure heavy phase) and the uptake phase (concentration phase—light phase).

After two phases with stable volumes have formed in the separating funnel, the light (upper) phase (concentration phase—light phase) and heavy (lower) phase (equilibrating solution) are separated and recovered separately.

At the same time the phase system E with purified protein is prepared. This is homogeneously mixed with a stirrer.

The interphase (diameter of heavy-phase weir—diameter of light-phase weir) is regulated to 2 mm by the incorporation of a heavy-phase weir.

Then the equilibrating solution is introduced into the centrifugal extractor through the heavy-phase inlet by means of a pump at a high flow rate; this serves to equilibrate the centrifugal extractor. For this purpose, the centrifuge flask is accelerated to a rotation speed of between 2800 revolutions per minute (rpm). When only the added equilibrating solution is flowing out of the heavy-phase outlet of the centrifugal extractor, phase system D (with cell culture) can then be fed in through the heavy-phase inlet at the same pump flow and rotation speed.

Light phase (PEG phase) with the target protein is then obtained through the light phase outlet, while the host cells, DNA and fragments thereof accumulate in the heavy phase.

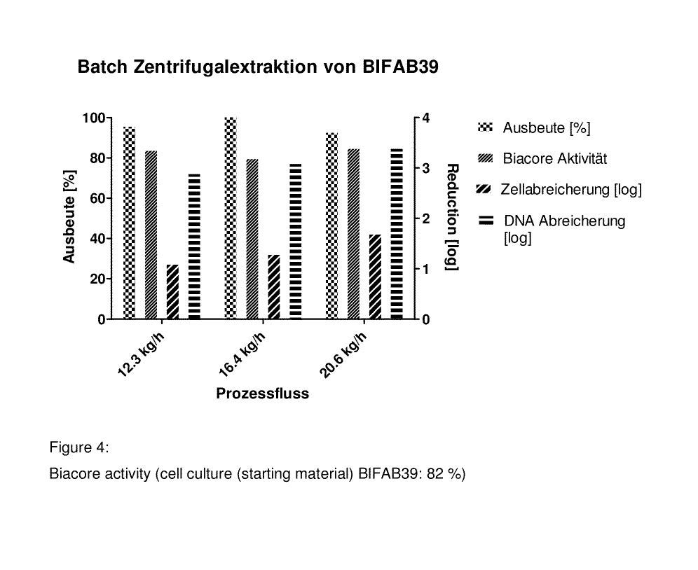

FIG. 4 shows a measurement of the number of CHO host cells, DNA content and the Biacore activity in the light phase after extraction in the centrifugal extractor at different process flows.

TABLE 3
Preparation of phase system D

| | |
|---|---|
| ATPS System [g] | 3000 |
| Stock solutions | |
| phosphate pH 6%[w/w] | 40 |
| PEG400%[w/w] | 100 |
| Protein content of cell culture BIFAB39 [g/L] | 1.53 |
| System | |
| PEG400%[w/w] | 23 |
| PO4 pH 6%[w/w] | 14 |
| NaCl %[w/w] | 0 |
| Protein content of system [g/L] | 0.3 |
| Initial weight | |
| phosphate pH 6%[g] | 1050 |
| PEG400%[g] | 690 |
| NaCl [g] | 0 |
| cell culture [g] | 600 |
| H₂O [g] | 660 |

4^{th} experiment: Concentration of protein (MAB) by process strategy

The centrifugal extractor can be used to concentrate target protein by aqueous two-phase extraction. First of all a phosphate stock solution is prepared. Then phase system A is prepared by weighing to the correct concentrations, as described above. Phase system A with the equilibrating solution of the centrifugal extractor (pure heavy phase) is placed in a separating funnel. After two phases with stable volume have formed in the separating funnel, the light/upper and heavy/lower phase (equilibrating solution) are separated and recovered separately.

Using the same method, phase system E with purified protein is prepared (cf. Table 5). It is homogeneously mixed with a stirrer.

TABLE 5
Preparation of phase system E

| | |
|---|---|
| ATPS System [g] | 5000 |
| Stock solutions | |
| Phosphate pH 6%[w/w] | 40 |
| PEG400%[w/w] | 100 |
| Protein [g/L] | 28.7 |
| System | |
| PEG400%[w/w] | 31 |
| PO4 pH 6%[w/w] | 1.25 |
| NaCl %[w/w] | 3.3 |
| Protein [g/L] | 1 |
| Initial weight | |
| phosphate pH 6%[g] | 3875 |
| PEG400%[g] | 62.5 |
| NaCl [g] | 165 |
| Protein [g] | 174.22 |
| H2O [g] | 723.28 |

The interphase in the centrifugal extractor (diameter of heavy-phase weir—diameter of light phase weir) is regulated to 2 mm by incorporation of a heavy-phase weir. Then, 500 g of the equilibrating solution is pumped into the centrifugal extractor through the heavy phase inlet by means of a pump at 10 L/H (litres per hour). To do this, the centrifuge flask is accelerated to a rotation speed of 3000 RPM.

When only the added equilibrating solution is still flowing out of the heavy phase outlet of the centrifugal extractor, the heavy phase—phase system E—is fed in through the heavy phase inlet and the light phase (take-up phase) is fed in through the light-phase inlet at different pumping rates (for process strategies cf. Table 6). Light phase (PEG phase) with the concentrated target protein is then obtained from the light phase outlet.

TABLE 6
Process control in the centrifugal extractor for concentration
(by means of the flow ratio of the light and heavy phase pump)

| | Pump flow | Pump flow | |
|---|---|---|---|
| | Heavy phase | Light phase | Flow ratio |
| Strategy | litres per hour | litres per hour | heavy to light phase |
| process control 1 | 4.3 | 4.3 | 1.0 |
| process control 2 | 10.0 | 4.3 | 2.3 |
| process control 3 | 20.0 | 4.3 | 4.7 |
| process control 4 | 13.1 | 1.4 | 9.4 |

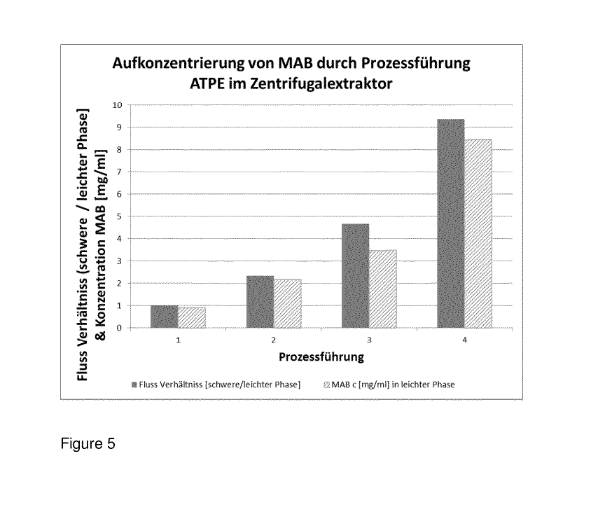

For results see FIG. 5.

Definitions of Non-SI Units:

RPM=revolutions per minute (rpm) of the centrifuge, for example

% w/w=wt. %, percent by weight, proportion by mass

### BIBLIOGRAPHY

1. Wurm. Nature Biotechnology. 2004, 22 (1393-1398).

2. Liu et al. mAbs. mAbs 2:5, 480-499; September/October 2010; ©2010 Landes Bioscience, 2010, Volumne 2 Issue 5 (480-499).

3. Pramanik et al. Characterization of Impurities and Degradants Using Mass Spectrometry. 2011.

4. Walsh. Drug Discovery Today. 2010, Bd. 15, 17-18 (773-780).

5. Shukla et al. Biopharm International. 2008, Bd. Volume 21, Issue 5.

6. Labrou et al. J Biotechnol. 1994, Bd. 36, 2 (95-119).

7. Abhinav et al. Trends in Biotechnology. 2010, Bd. 28, 5 (253-261).

8. Chmiel. Bioprozesstechnik. Spektrum Akademischer Verlag. 2006.

9. Gottschalk et al. Process Scale Purification of Antibodies. 2008.

10. Brekke et al. Current Opinion in Pharmacology. 2003, 5 (544-550).

11. Voitl et al. Journal of Chromatography A. 2010, 1217 (5753-5760).

12. Bensch et al. Chemical Engineering Science. 2007, 7 (2011-2021).

13. Bora et al. Separation and Purification Technology. 2005, Bd. Volume 45, Issue 2 (153-156).

14. Kepka et al. Journal of Chromatography A. 2004, Bd. Volume 1057, Issues 1-2 (115-124).

15. Asenjo et al. Journal of Chromatography A. 1994, Bd. Volume 668, Issue 1 (129-137).

16. Azevedo et al. Trends in Biotechnology. 2009, Bd. Volume 27, Issue 4 (Pages 240-247).

17. Rosa et al. Journal of Biotechnology. 2009, Bd. Volume 139, Issue 4 (306-313).

18. Albertsons, P. A. (1971) Partition of Cell Particles and Macromolecules, 2nd edn. Almqvist and Wiksell, Stockholm and Wiley, New York .

19. Oelmeier and Hubbuch, Journal of Chromatography A 2012, Bd. Volume 1252 (104-114).

1. Method for the selective purification and concentration of immunoglobulins or other proteins by means of an aqueous two-phase system, comprising the steps of:
a. preparing a cell culture or a cell culture supernatant which contains the target protein;
b. converting the cell culture or the cell culture supernatant into an aqueous two-phase system by the addition of a polymer and at least one salt, or two polymers in a suitable concentration;
c. thoroughly mixing the two-phase system to produce a dispersion;
d. separating the heavy and light phase in a centrifugal extractor;
e. recovering the target protein from the light phase.

2. Method for the selective purification and accumulation and additional concentration of immunoglobulins or other proteins by means of an aqueous two-phase system, comprising the steps of:
a. preparing a cell culture or a cell culture supernatant which contains the target protein;
b. preparing a light phase of the aqueous two-phase system by adding a polymer and at least one salt, or two polymers, to an aqueous medium in a suitable concentration;
c. converting the cell culture or the cell culture supernatant into the heavy phase of the aqueous two-phase system by the addition of a polymer and at least one salt, or two polymers in a suitable concentration;
d. separately feeding the light and heavy phases into the mixing chamber of the centrifugal extractor at different flow rates, the ratio of the flow rates corresponding to the desired concentration ratio;
e. thoroughly mixing the two-phase system to produce a dispersion;
f. separating the heavy and light phase in the centrifugal extractor;
g. recovering the target protein from the light phase.

3. Method according to claim 1, wherein the two-phase system contains polyethyleneglycol with a molecular weight of between 200 and 1000 g/mol.

4. Method according to claim 3, wherein the polyethyleneglycol has a molecular weight of between 400 and 600 g/mol.

5. Method according to claim 3, wherein the polyethyleneglycol is present in the two-phase system in a concentration of 18 to 35 wt. %.

6. Method according to claim 1, wherein the salt or one of the salts is a phosphate salt.

7. Method according to claim 1, wherein the pH of the two-phase system is between 5 and 7.

8. Method according to claim 1, wherein the centrifugal extractor is pre-equilibrated with the heavy phase.

9. Method according to claim 1, wherein the two phases are separated with a centrifugal acceleration of 40-100% of the maximum possible g-force in the apparatus used.

10. Method according to claim 1, wherein the two phases are separated with a centrifugal acceleration of 90-500×g.

11. Method according to claim 1, wherein the density ratio of the two phases is at least 1.06.

12. Method according to claim 2, wherein the two-phase system contains polyethyleneglycol with a molecular weight of between 200 and 1000 g/mol.

13. Method according to claim 12, wherein the polyethyleneglycol has a molecular weight of between 400 and 600 g/mol.

14. Method according to claim 12, wherein the polyethyleneglycol is present in the two-phase system in a concentration of 18 to 35 wt. %.

15. Method according to claim 2, wherein the salt or one of the salts is a phosphate salt.

16. Method according to claim 2, wherein the pH of the two-phase system is between 5 and 7.

17. Method according to claim 2, wherein the centrifugal extractor is pre-equilibrated with the heavy phase.

18. Method according to claim 2, wherein the two phases are separated with a centrifugal acceleration of 40-100% of the maximum possible g-force in the apparatus used.

19. Method according to claim 2, wherein the two phases are separated with a centrifugal acceleration of 90-500×g.

20. Method according to claim 2, wherein the density ratio of the two phases is at least 1.06.
